(19) United States
(12) Patent Application Publication
(10) Pub. No.: US 2024/0316187 A1
(43) Pub. Date: Sep. 26, 2024

(54) PROTEINS IDENTIFIED FROM BARN DUST EXTRACT FOR THE PREVENTION AND TREATMENT OF DISEASES

(71) Applicant: Helmholtz Zentrum München - Deutsches Forschungszentrum für Gesundheit und Umwelt (GmbH), Neuherberg (DE)

(72) Inventors: Erika VON MUTIUS, Garmisch-Partenkirchen (DE); Bettina RANKL, Gilching (DE); Franz BRACHER, Gröbenzell (DE); Christoph MÜLLER, München (DE); Alesia WALKER, Oberschleissheim (DE); Stefanie HAUCK, München (DE); Juliane MERL-PHAM, München (DE); Heiko ADLER, München (DE); Ali Önder YILDIRIM, München (DE); Michael SATTLER, München (DE); André SANTOS DIAS MOURÃO, München (DE); Jan BORGGRÄFE, München (DE); Patrick David O'CONNOR, München (DE); Oliver PLETTENBURG, Hannover (DE)

(21) Appl. No.: 18/294,951

(22) PCT Filed: Aug. 3, 2022
(86) PCT No.: PCT/EP2022/071830

§ 371 Date: Feb. 2, 2024

(30) Foreign Application Priority Data
Aug. 3, 2021 (EP) 21189353.2

(51) Int. Cl. A61K 39/36; A61K 39/35; A61P 11/06; A61P 37/08

(52) U.S. Cl. CPC A61K 39/36; A61K 39/35; A61P 11/06; A61P 37/08

(57) ABSTRACT

The present invention relates to a composition comprising proteins identified in barn dust extract or peptides derived from one of the proteins. The composition is useful in the prevention or treatment of a disease.

### CROSS-REFERENCE TO RELATED APPLICATIONS

This application claims the benefit of priority of European patent application no. 21 189 353.2 filed 3 Aug. 2021, the contents of which are being hereby incorporated by reference in their entirety for all purposes.

### FIELD OF THE INVENTION

The present invention relates to a composition comprising one or several proteins identified from barn dust extract, as well as the use thereof in the treatment or prevention of disease.

### BACKGROUND

Allergy has developed into a major health concern in Europe with over 80 million people affected by some form of allergic disease and around 30 million people suffering from asthma. About 50% of all children (in some areas even 70%) have a positive allergy test to ‘normal’ exposures such as house dust, animals, pollen and food. The human immune system has become less able to tolerate these natural exposures and instead reacts with immunoglobulin E (IgE) antibodies, therefore developing allergic sensitization. These overshooting immune responses result in allergic airway disease such as hay fever and allergic asthma. The prevalence of hay fever is 20% among school children and 30% among teenagers. About 10% of children suffer from asthma. Most allergic patients develop a chronic course spreading into adolescence and adult age which results in significant health threats and severe limitations of activities and quality of life.

A way to prevent the new onset of asthma, hay fever and allergic sensitization is yet to be found, however there is compelling and consistent evidence across more than 30 studies worldwide that children growing up in a farming environment rich in microbial exposures are better protected from developing asthma, allergic rhinitis and atopic sensitization.

In the ALEX study the differences between farm and nonfarm children were: 1.4% versus 11.8% asthma, 3.2% versus 16% hay fever, 12.4% versus 32.9% allergic sensitization. This protection is sustained into adult life (von Mutius E and Vercelli D. Farm living: Effects on childhood asthma and allergy. Nat Rev Immunol. 2012; 10: 861-868 (2010) PMID: 21060319).

The farm effect on asthma can be explained by the child's early life contact to farm animals, mostly cattle (Illi S, Depner M, Genuneit J, et al. Protection from childhood asthma and allergy in Alpine farm environments—the GABRIEL Advanced Studies. J Allergy Clin Immunol 2012; 129:1470-7 e6; Loss G J, Depner M, Hose A J, et al. The Early Development of Wheeze. Environmental Determinants and Genetic Susceptibility at 17q21. Am J Respir Crit Care Med 2016; 193:889-97). These findings suggest that exposures encountered in animal sheds, in particular cattle stables, play a major role. Importantly, the epidemiological findings have been translated into experimental studies in mice thereby adding biological validity to these observations. In two independent studies, dust from a Bavarian cow shed (Schuijs M J, Willart M A, Vergote K, et al. Farm dust and endotoxin protect against allergy through A20 induction in lung epithelial cells. Science 2015; 349:1106-10) and aqueous extract of cow shed dust collected in the ALEX study (Peters M, Kauth M, Schemer 0, et al. Arabinogalactan isolated from cowshed dust extract protects mice from allergic airway inflammation and sensitization. J Allergy Clin Immunol 2010; 126:648-56 el-4) were administered in aerosolized form (Peters M, Kauth M, Schemer 0, et al. Arabinogalactan isolated from cowshed dust extract protects mice from allergic airway inflammation and sensitization. J Allergy Clin Immunol 2010; 126:648-56 el-4) or intranasally (Schuijs M J, Willart M A, Vergote K, et al. Farm dust and endotoxin protect against allergy through A20 induction in lung epithelial cells. Science 2015; 349:1106-10) in ovalbumin (OVA)-induced allergic asthma model (Peters M, Kauth M, Schemer 0, et al. Arabinogalactan isolated from cowshed dust extract protects mice from allergic airway inflammation and sensitization. J Allergy Clin Immunol 2010; 126:648-56 el-4) or house dust mite (Schuijs M J, Willart M A, Vergote K, et al. Farm dust and endotoxin protect against allergy through A20 induction in lung epithelial cells. Science 2015; 349:1106-10) models of allergic asthma. In all studies this prophylactic treatment resulted in dramatically reduced airway hyperresponsiveness, suppressed eosinophilia and reduced levels of Immunoglobulin E (IgE), all hallmarks of allergic asthma.

In our previous research, we found that not all farms are created equal in terms of their asthma- and allergy-protective properties. The PARSIFAL cross-sectional survey asked farmers whether they performed livestock farming, agriculture (cultivation of grain), or both, and assessed the animal species kept on the farm (cattle, pigs, poultry, horses, sheep, goats, hares, and rabbits). Asthma risk was significantly reduced by a child's frequent stay in animal sheds, her/his involvement in having activities, and the additional cultivation of grain on an animal farm, suggesting that exposure to animals and plant material (straw and hay) was essential for asthma protection (Ege M J, Frei R, Bieli C, et al. Not all farming environments protect against the development of asthma and wheeze in children. J Allergy Clin Immunol. 2007 May; 119(5):1140-7.). Especially children exposed to cow sheds early in life are at significantly lower risk of developing hay fever, allergic sensitization, asthma and early symptoms of asthma (wheeze) than children not growing up on farms. In contrast, children growing up on sheep farms were at higher risk of current wheeze and allergic asthma. The role of animal and plant contact in asthma protection was corroborated by the GABRIEL cross-sectional survey, which included a refined and detailed farm exposure matrix. In that study, the child's contact to cow sheds in which straw and/or hay were used was significantly associated with asthma protection.

Allergy therapies must often be administered over years and decades, and the application of newly developed biologicals results in exploding costs, therefore there is a need of developing preventive cures as they represent the only true long-term solution. Therefore, the technical problem underlying the present invention is the provision of further compositions and therapies for allergy development, in particular preventive therapies.

### SUMMARY OF THE INVENTION

The present invention relates to a composition for use in the prevention or treatment of a disease, comprising at least one protein or a peptide derived from one of these proteins having an amino acid sequence at least 90% identical to any one of SEQ ID NO:1-13. The composition can be used for the treatment or prevention of a disease that is selected from the group consisting of an allergic disease, a chronic inflammatory disease, and an autoimmune disease. Specifically, the disease can be selected from the group consisting of hay fever, food allergy, asthma, urticaria, neurodermitis, atopy, including atopic sensitisation and atopic dermatitis, contact eczema, psoriasis, diabetes type 1 or 2, multiple sclerosis, rheumatoid arthritis, diseases of the thyroid gland, including Hashimoto thyreoditis and Graves disease, preferably selected from the group consisting of atopy, including atopic sensitisation and atopic dermatitis, asthma and hay fever.

The composition for the use of the invention may comprises 2, 3, 4, 5, 6, 7, 8, 9, 10, 11, 12 or all of the proteins or peptides derived from these proteins having an amino acid sequence at least 90% identical to of any one of SEQ ID NO:1-13. The proteins or peptides derived from these proteins in the composition of the invention may be at least 95%, at least 99% or are 100% identical to any one of SEQ ID NO:1-13. Preferably, a protein or peptide derived from these proteins in the composition of the invention is at least 90%, at least 95%, at least 99% or 100% identical to SEQ ID NO:2. Preferably, a protein or peptide derived from these proteins in the composition of the invention is at least 90%, at least 95%, at least 99% or 100% identical to SEQ ID NO:7.

In one embodiment, the composition comprises a combination of SEQ ID NO:1 together with at least one of SEQ ID NO:2-13. Alternately, the composition can comprise a combination of SEQ ID NO:1 and SEQ ID NO:2, optionally with one or more of SEQ ID NO:3, SEQ ID NO:7 or SEQ ID NO:9. The composition can comprise a combination of SEQ ID NO:1 and SEQ ID NO:7, optionally with one or more of SEQ ID NO:1, SEQ ID NO:2 or SEQ ID NO:9.

The composition may comprise a combination of SEQ ID NO:2 together with at least one of SEQ ID NO:1, 3-13. The composition may comprise a combination of SEQ ID NO:2 and SEQ ID NO:1, optionally with one or more of SEQ ID NO:3, SEQ ID NO:7 or SEQ ID NO:9. The composition may comprise a combination of SEQ ID NO:2 and SEQ ID NO:7, optionally with one or more of SEQ ID NO:1, SEQ ID NO:3 or SEQ ID NO:9.

The composition may comprise a combination of SEQ ID NO:7 together with at least one of SEQ ID NO:1-6, 8-13. The composition may comprise a combination of SEQ ID NO:7 and SEQ ID NO:1, optionally with one or more of SEQ ID NO:2, SEQ ID NO:3 or SEQ ID NO:9. The composition may comprise a combination of SEQ ID NO:7 and SEQ ID NO:2, optionally with one or more of SEQ ID NO:1, SEQ ID NO:3 or SEQ ID NO:9.

The composition may comprise a combination of a protein or peptide having at least 90% identity to SEQ ID NO:2 together with a protein or peptide having at least 90% identity to SEQ ID NO:7. The composition may comprise a combination of a protein or peptide having at least 95% identity to SEQ ID NO:2 together with a protein or peptide having at least 95% identity to SEQ ID NO:7. The composition may comprise a combination of a protein or peptide having at least 98% identity to SEQ ID NO:2 together with a protein or peptide having at least 98% identity to SEQ ID NO:7. The composition may comprise a combination of a protein or peptide having at least 99% identity to SEQ ID NO:2 together with a protein or peptide having at least 99% identity to SEQ ID NO:7.

The composition for the use of the invention can be in the form of a solution, an aerosol, a suspension, a lyophilisate, a powder, a tablet, a dragee, suppository, enema, ointment, creme or plaster. The composition may be for nasal, inhalative, oral, conjunctival, dermal, subcutaneous, intraarticular, intraperitoneal, rectal, or vaginal administration.

In addition, the composition for the use of the invention may include ligands bound to at least one protein or peptides derived from the proteins having an amino acid sequence at least 90%, at least 95%, at least 99% or 100% identical to any one of SEQ ID NO:1-13.

The invention also relates to a pharmaceutical composition comprising at least one protein or peptides derived from the proteins having an amino acid sequence at least 90%, at least 95%, at least 99% or 100% identical to any one of SEQ ID NO:1-13, in some cases the composition comprises 2, 3, 4, 5, 6, 7, 8, 9, 10, 11, 12 or all of the proteins or peptides derived from the proteins having an amino acid sequence at least 90%, at least 95%, at least 99% or 100% identical to of any one of SEQ ID NO:1-13. In some cases, the proteins or peptides derived from the proteins are at least 95% or are 100% identical to any one of SEQ ID NO:1-13.

The proteins or peptides derived from the proteins in the composition of the invention may be at least 95%, 99% or are 100% identical to any one of SEQ ID NO:1-13.

In one embodiment, the pharmaceutical composition comprises a combination of SEQ ID NO:1 together with at least one of SEQ ID NO:2-13. Alternately, the pharmaceutical composition can comprise a combination of SEQ ID NO:1 and SEQ ID NO:2, optionally with one or more of SEQ ID NO:3, SEQ ID NO:7 or SEQ ID NO:9. The pharmaceutical composition can comprise a combination of SEQ ID NO:1 and SEQ ID NO:7, optionally with one or more of SEQ ID NO:1, SEQ ID NO:2 or SEQ ID NO:9.

The composition may comprise a combination of SEQ ID NO:2 together with at least one of SEQ ID NO:1, 3-13. The composition may comprise a combination of SEQ ID NO:2 and SEQ ID NO:1, optionally with one or more of SEQ ID NO:3, SEQ ID NO:7 or SEQ ID NO:9. The composition may comprise a combination of SEQ ID NO:2 and SEQ ID NO:7, optionally with one or more of SEQ ID NO:1, SEQ ID NO:3 or SEQ ID NO:9.

The composition may comprise a combination of SEQ ID NO:7 together with at least one of SEQ ID NO:1-6, 8-13. The composition may comprise a combination of SEQ ID NO:7 and SEQ ID NO:1, optionally with one or more of SEQ ID NO:2, SEQ ID NO:3 or SEQ ID NO:9. The composition may comprise a combination of SEQ ID NO:7 and SEQ ID NO:2, optionally with one or more of SEQ ID NO:1, SEQ ID NO:3 or SEQ ID NO:9.

The composition may comprise a combination of a protein or peptide having at least 90% identity to SEQ ID NO:2 together with a protein or peptide having at least 90% identity to SEQ ID NO:7. The composition may comprise a combination of a protein or peptide having at least 95% identity to SEQ ID NO:2 together with a protein or peptide having at least 95% identity to SEQ ID NO:7. The composition may comprise a combination of a protein or peptide having at least 98% identity to SEQ ID NO:2 together with a protein or peptide having at least 98% identity to SEQ ID NO:7. The composition may comprise a combination of a protein or peptide having at least 99% identity to SEQ ID NO:2 together with a protein or peptide having at least 99% identity to SEQ ID NO:7.

The pharmaceutical composition for the use of the invention can be in the form of a solution, an aerosol, a suspension, a lyophilisate, a powder, a tablet, a dragee, suppository, enema, ointment, creme or plaster. The pharmaceutical composition may be for nasal, inhalative, oral, conjunctival, dermal, subcutaneous, intraarticular, intraperitoneal, rectal, or vaginal administration.

In addition, the pharmaceutical composition for the use of the invention may include a ligand bound to the at least one protein or peptide derived from one of the proteins having an amino acid sequence at least 90%, at least 95%, at least 99% or 100% identical to any one of SEQ ID NO:1-13.

### BRIEF DESCRIPTION OF THE DRAWINGS

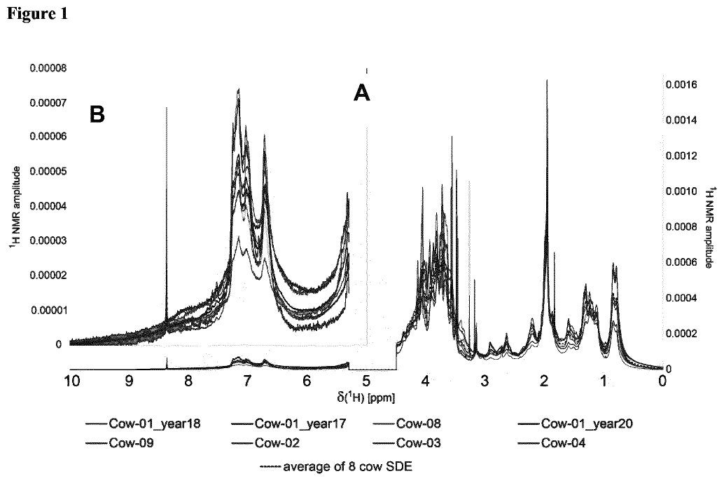

### FIG. 1

Area-normalized ¹H NMR spectra (800 MHz, D₂O) of eight cow shed dust extract (SDE) aqueous extracts and computed average ¹H NMR spectrum (dotted line). A entire spectrum, B section of aliphatic peptide side chains, OCH, and CCH units, C section of unsaturated and aromatic units. All cow-derived BD show closely related curvature of ¹H NMR spectra, corroborating rather congruent structural main features that primarily originate from amino acid side chains (δ_{H}˜0.5-4.5 ppm for aliphatic, and δ_{H}˜ 6.5-8.2 ppm for aromatic side chains) and from carbohydrates (δ_{H}˜ 3.2-4.5 ppm). Natural variability of SDE as expressed by variance of ¹H NMR resonance amplitude reflects different relative abundance of main structural units of SDE. This likely results from intrinsic source heterogeneity within the original solid comprised of minerals and a complex mixture of organic molecules, and nutrient, temperature and water dependent microbial processing. The considerable variance of protein/carbohydrate ratios in all cow-SDE peaks against a dominating presence of glycoproteins and absence of relevant polymethylene-related ¹H NMR resonances (δ_{H}˜ 1.2 ppm) speaks against a major decisive role of lipopolysaccharides (LPS).

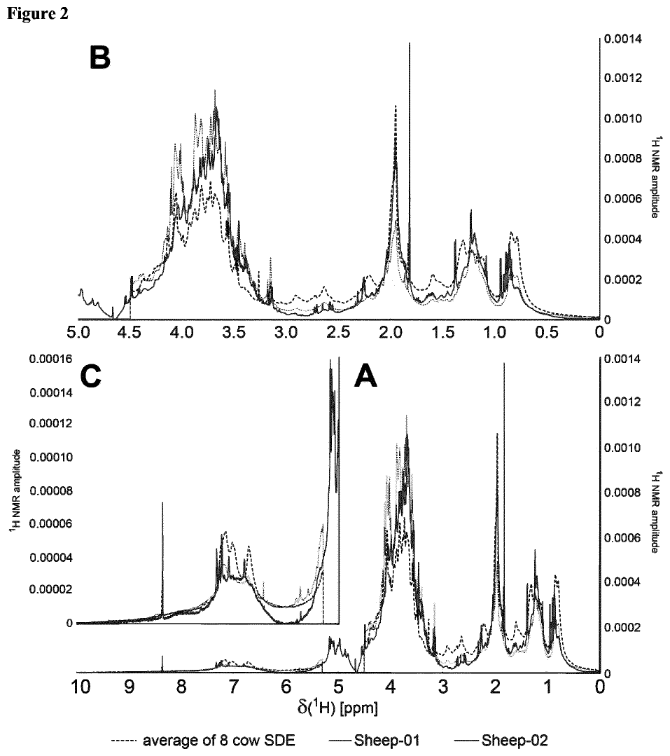

### FIG. 2

Area-normalized ¹H NMR spectra (800 MHz, D₂O) of two sheep SDE extracts, compared with computed average of eight cow-related BD extracts. A entire spectrum, B section of aliphatic peptide side chains, OCH, and CCH units, C section of unsaturated and aromatic units. SDE HM_2021_110521 is shown also near the water resonance (δ_{H}˜ 4.7 ppm) to demonstrate presence of anomeric O₂CH units (δ_{H}˜ 4.7-5.3 ppm) in a richly diverse set of carbohydrates.

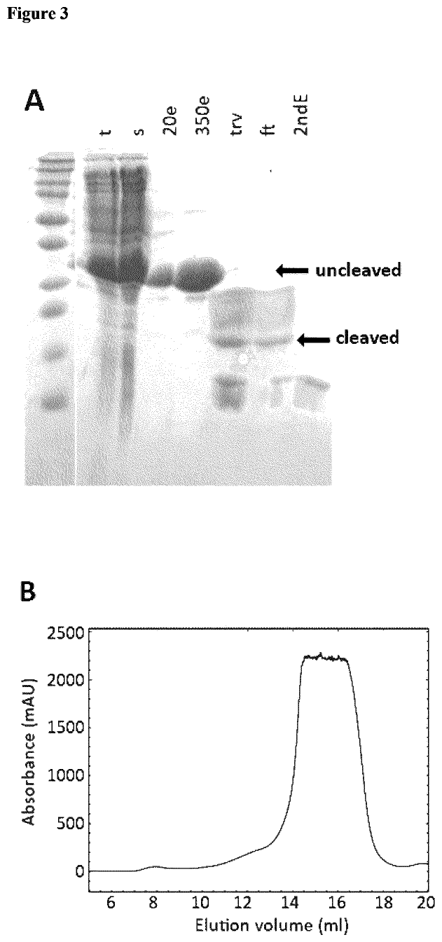

### FIG. 3

Protein expression of bOBP and Bos d2. A: Exemplary SDS-PA purification gel of pOBP. (t) lysate, (s) supernatant, (20e) 1^{st} IMAC wash fraction, (350e)₁^{st} IMAC elution, (trv) after TEV cleavage, (ft) 2^{nd} IMAC flow-through, (2ndE) 2^{nd} IMAC elution. Arrows indicate pOBP with cleaved/uncleaved His-Trx tag. B: SEC profile of purified pOBP. C+D: SEC profile of purified Bos d2 shows several elution peaks (D). Analysis of the elution fractions by SDS-PAGE shows insufficient TEV cleavage of His-Trx-Bos d2.

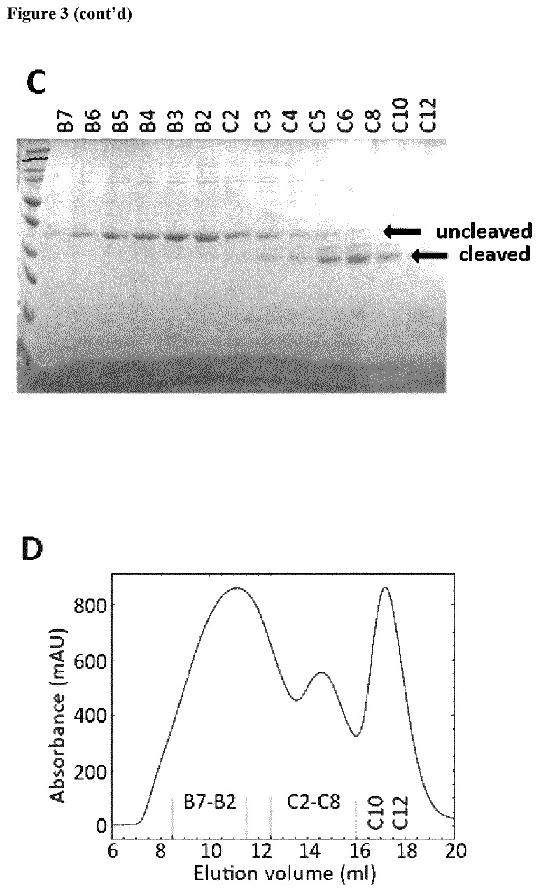

### FIG. 4:

NMR binding studies. Overlay of ¹H, ⁵N-HSQC spectra of ¹⁵N-bOBP alone and in presence of 5- and 10-fold excess of (E)-12-oxoheptadec-10-enoic acid. Arrow and inlet show CSP of exemplary peak. Arrows and inlets show CSP of exemplary peaks. Notably, affected peaks are located in similar regions in the titration experiment, indicating similar binding sites for (E)-12-oxoheptadec-10-enoic acid and extract component(s).

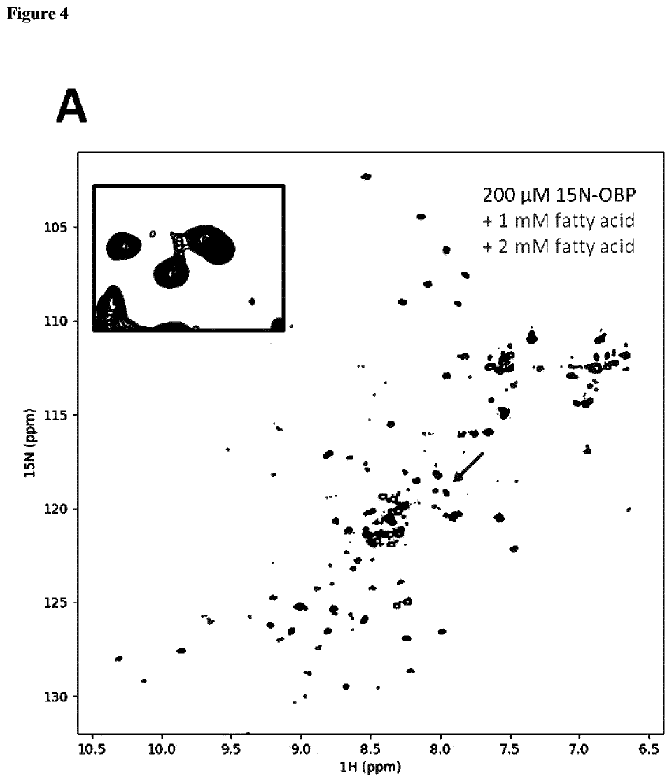

### FIG. 5:

NKfB induction in HEK-hTLR2. Dotted line: PBS/DMSO (negative control); Pam3CSK4=positive control; Farm dust (Hec 20)=Cow shed dust extract>10 kDa autoclaved; Bosd2+FA=His-Trx-Bos d2+€-12-oxoheptadec-10-enaic acid; OBP+FA=bOBP+€-12-oxoheptadec-10-enaic acid; OBP=bOBP; FA=€-12-oxoheptadec-10-enaic acid.

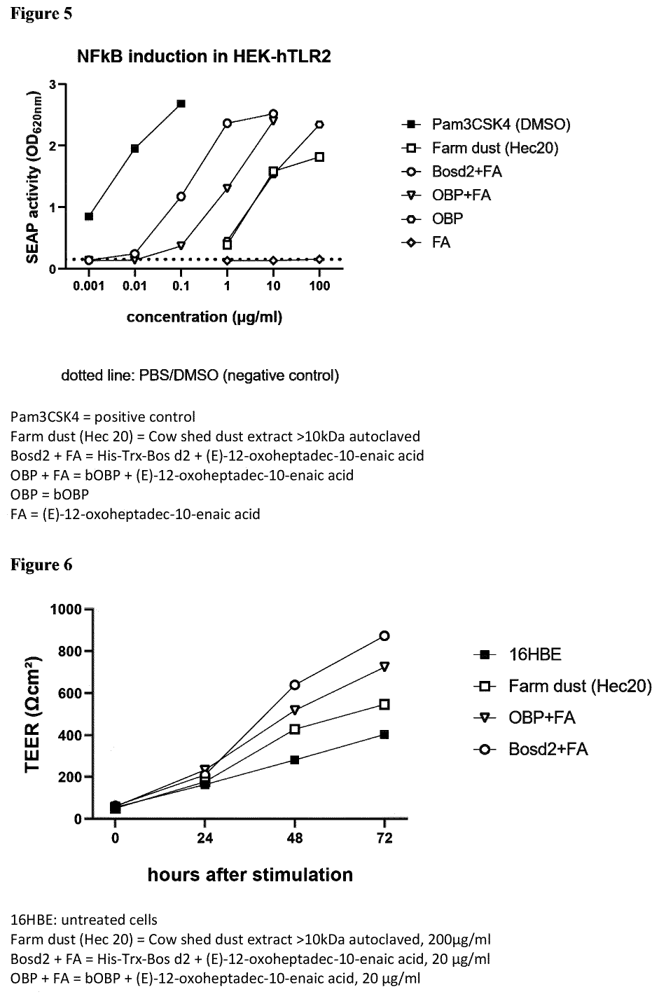

### FIG. 6:

TEER Assay: 16HBE: untreated cells; Farm dust (Hec 20)=Cow shed dust extract>10 kDa autoclaved, 200 μg/ml; Bosd2+FA=His-Trx-Bos d2+(E)-12-oxoheptadec-10-enaic acid, 20 μg/ml; OBP+FA=bOBP+(E)-12-oxoheptadec-10-enaic acid, 20 μg/ml.

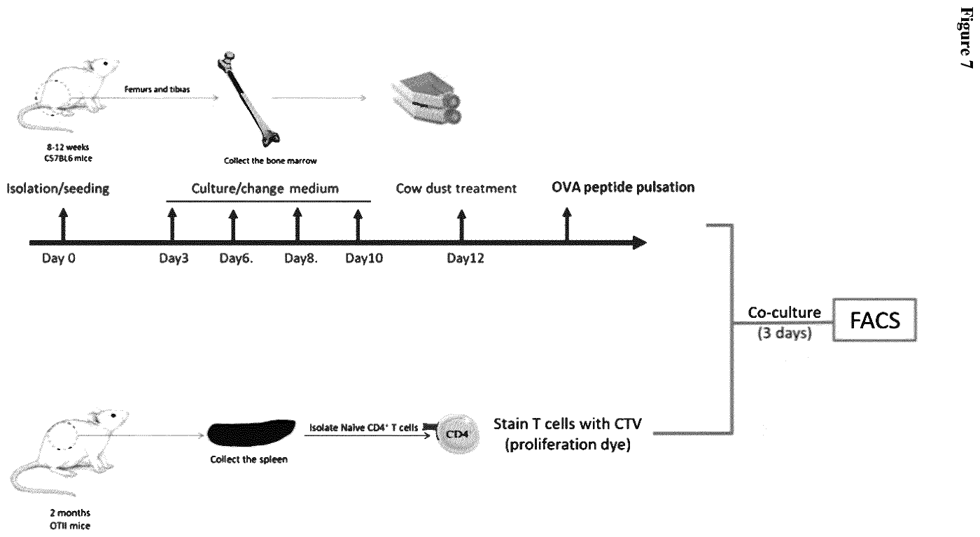

### FIG. 7:

Assay set up of DC/T-cell interaction assay.

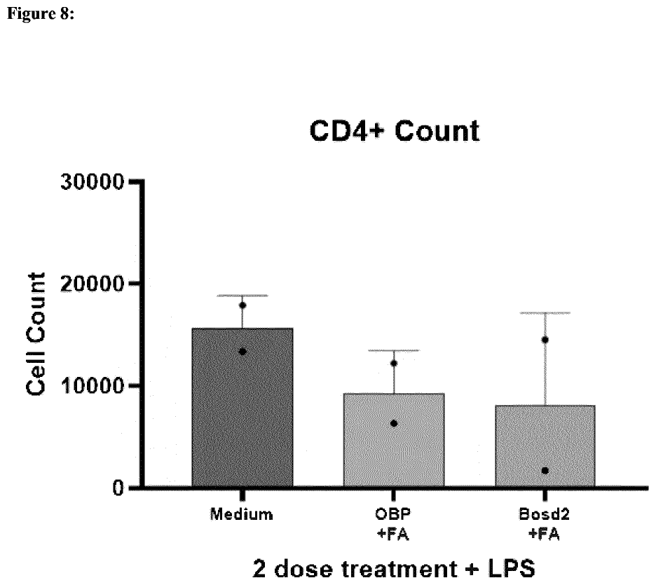

### FIG. 8:

Effect of lipoprotein candidates on DC T-cell interaction: OVA pulsed BMDCs enhanced OT-II CD4 T cell number (Medium), which was prevented by pre-treatment of BMDCs with, Bos d2+FA and and OPB+FA. CD4+ count: For each sample 10400 counting beads were added to tube and 5000 beads were counted for recording. CD4+ count indicates the amount of CD4+ cells contained in the volume needed to count 5000 beads. (FA: (E)-12-oxoheptadec-10-enaic acid).

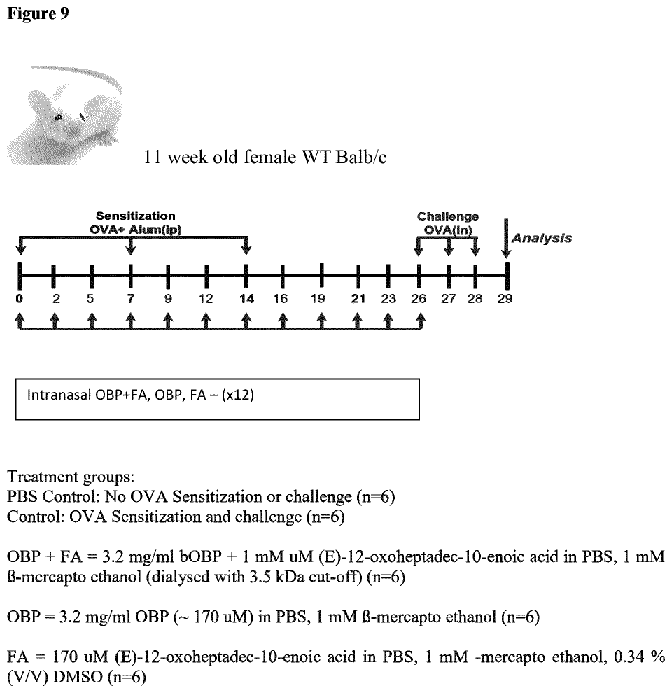

### FIG. 9:

Assay set up of Example 5.

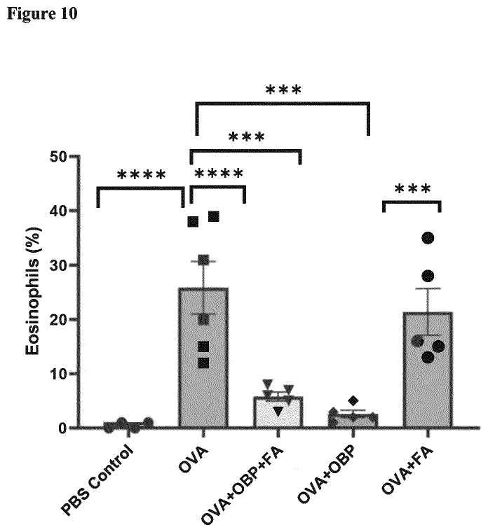

### FIG. 10:

Intranasal application of OBP+FA and OBP prevented allergic asthma in mice sensitized with OVA as determined by Bronchoalveolar lavage (BAL) eosinophil numbers. Control mice were sham sensitized to PBS. Error bars reflect SEM. P values reflect one-way ANOVA, *: p value<0.05, **: p value<0.01; (FA: (E)-12-oxoheptadec-10-enaic acid).

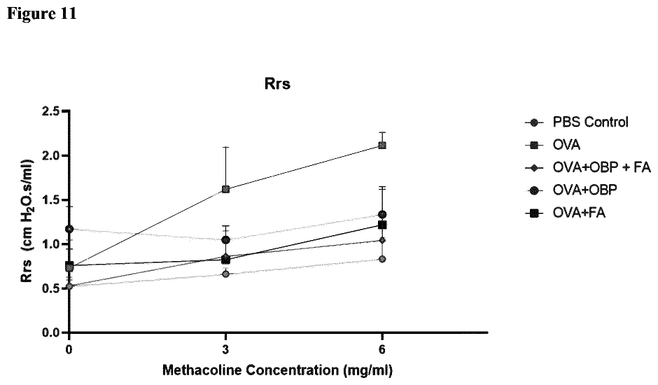

### FIG. 11:

Intranasal application of OBP+FA and OBP prevented allergic asthma in mice sensitized with OVA as determined by measurement of AHR to increasing doses of methacholine. Control mice were sham sensitized to PBS. Error bars reflect SEM. (FA: (E)-12-oxoheptadec-10-enaic acid).

### DETAILED DESCRIPTION

The present invention is based on the surprising finding that a core set of proteins is present in barn dust extracts from cow sheds that is absent from barn dust extracts from sheep sheds.

In our previous work, we found that fractions of autoclaved stable dust extract≥10 kDa from cow sheds had a protective effect on mice (WO 2020/120724). Therefore we aimed to elucidate the active components in cow dust extracts. Aqueous extracts of sheep barn dusts collected and prepared in identical ways as cow barn extracts and were characterized by nuclear magnetic resonance spectroscopy (NMR). One and two dimensional ¹H and ¹³C NMR spectra of cow shed dust extracts (SDE) identified protein and carbohydrate related chemical environments as major constituents of the size fractions≥10 kDa. ¹H detected NMR spectra showed dominance of proteins and carbohydrates in SDE, explaining>95% of overall NMR integral (FIG. 1).

We prepared extract fractions with molecules≥10 kDA from cow and sheep dust extract. In addition the extracts were autoclaved to exclude the potential effect of live microorganisms. As compared to cow dust extracts autoclaved sheep dust extracts≥10 kDa did not prevent experimental allergic asthma, suggesting that a) live microorganisms are not important for the effect, b) substances contained in the fraction≥10 kDa in cow dust extracts carry the protective effect, as shown previously in WO 2020/120724.

To get further information about the differences in the composition of these extracts we assessed the relative proportions of carbohydrate and peptide related structures by NMR. Commonly, SDE derived from sheep showed consistently higher relative proportions of carbohydrates and lower proportions of peptides/proteins than cow SDE as can be seen from FIG. 2.

Given the differences in peptide content between sheep and cow shed dust extracts, we performed proteomics analysis of autoclaved dust extracts≥10 kDa from n=7 different cow and n=2 different sheep sheds, as described in Example 2. Three datasets were combined, resulting in a list of 1888 identified proteins, of which 264 proteins fulfilled the criteria to be quantified with at least 2 peptides in at least one of the three datasets and show an enrichment ratio cow/sheep of >5 for at least one cow dust sample.

Differential analysis revealed a set of proteins and peptides which were to a much larger degree (at least 5-fold) or only contained in cow dust extracts.

The set of proteins are displayed in the table below.

| SEQ | | | |
|---|---|---|---|
| ID | Accession | Name | Description/Function |
| 1. | P02769 | Serum albumin | Bos Taurus (Bovine) |
| | | BSA, | Binds water, Ca²⁺, Na⁺, K⁺, fatty acids, hormones, |
| | | Allergen Bos d6 | bilirubin and drugs. Its main function is the |
| | | | regulation of the colloidal osmotic pressure of blood. |
| | | | Major zinc transporter in plasma, typically binds about |
| | | | 80% of all plasma zinc (By similarity). Major calcium |
| | | | and magnesium transporter in plasma, binds |
| | | | approximately 45% of circulating calcium and |
| | | | magnesium in plasma (Probable). Potentially has more |
| | | | than two calcium-binding sites and might additionally |
| | | | bind calcium in a non-specific manner. The shared |
| | | | binding site between zinc and calcium at residue Asp- |
| | | | 272 suggests a crosstalk between zinc and calcium |
| | | | transport in the blood (Probable). The rank order of |
| | | | affinity is zinc > calcium > magnesium (Probable). |
| | | | Binds to the bacterial siderophore enterobactin and |
| | | | inhibits enterobactin-mediated iron uptake of E.coli, |
| | | | and may thereby limit the utilization of iron and |
| | | | growth of enteric bacteria such as E.coli. Does not |
| | | | prevent iron uptake by the bacterial siderophore |
| | | | aerobactin. |
| 2. | Q28133 | Allergen Bos d2 | Bos taurus (Bovine) |
| | | Dander major | Belongs to the calycin superfamily. Lipocalin family; |
| | | allergen BDA20 | Found exclusively in skin. Produced in sweat glands |
| | | | and transported to the skin surface |
| 3. | P79124 | Short palate, | Bos Taurus (Bovine) |
| | | lung and nasal | Lipopolysaccharide binding |
| | | epithelium | |
| | | carcinoma- | |
| | | associated | |
| | | protein 2A | |
| | | BSP30a, | |
| | | common | |
| | | salivary protein | |
| | | form a | |
| 4. | P80416 | Cystatin-A | Bos Taurus (Bovine) |
| | | Cystatin-A, | This is an intracellular thiol proteinase inhibitor. |
| | | Stefin-A | |
| 5. | P01083 | Alpha-amylase | Triticum aestivum (Wheat) |
| | | inhibitor 0.28 | |
| | | CIII | Belongs to the protease inhibitor I6 (cereal |
| | | WMAI-1 | trypsin/alpha-amylase inhibitor) family. |
| 6. | P79345 | NPC | Bos taurus (Bovine) |
| | | intracellular | Intracellular cholesterol transporter which acts in |
| | | cholesterol | concert with NPC1 and plays an important role in the |
| | | transporter 2 | egress of cholesterol from the lysosomal compartment. |
| | | EPV20 | Unesterified cholesterol that has been released from |
| | | Epididymal | LDLs in the lumen of the late endosomes/lysosomes is |
| | | secretory protein | transferred by NPC2 to the cholesterol-binding pocket |
| | | E1 | in the N-terminal domain of NPC1 (By similarity). |
| | | Niemann Pick | May bind and mobilize cholesterol that is associated |
| | | type C2 protein | with membranes. NPC2 binds cholesterol with a 1:1 |
| | | homolog | stoichiometry. Can bind a variety of sterols, |
| | | | including lathosterol, desmosterol and the plant |
| | | | sterols stigmasterol and beta-sitosterol (By |
| | | | similarity). The secreted form of NCP2 regulates |
| | | | biliary cholesterol secretion via stimulation of |
| | | | ABCG5/ABCG8-mediated cholesterol transport (By |
| | | | similarity). |
| 7. | P07435 | Odorant- | Bos Taurus (Bovine) |
| | | binding protein | This protein binds a wide variety of chemical |
| | | OBP | odorants. |
| | | Olfactory | Belongs to the calycin superfamily. Lipocalin family |
| | | mucosa | |
| | | pyrazine-binding | |
| | | protein | |
| 8. | P79105 | Protein S100- | Bos Taurus (Bovine) |
| | | A12 | S100A12 is a calcium-, zinc- and copper-binding |
| | | Calcium-binding | protein which plays a prominent role in the regulation |
| | | protein amniotic | of inflammatory processes and immune response. Its |
| | | fluid 1 | proinflammatory activity involves recruitment of |
| | | Calgranulin-C | leukocytes, promotion of cytokine and chemokine |
| | | Cornea- | production, and regulation of leukocyte adhesion and |
| | | associated | migration. Acts as an alarmin or a danger associated |
| | | antigen | molecular pattern (DAMP) molecule and stimulates |
| | | Extracellular | innate immune cells via binding to receptor for |
| | | newly identified | advanced glycation endproducts (AGER). Binding to |
| | | RAGE-binding | AGER activates the MAP-kinase and NF-kappa-B |
| | | protein | signaling pathways leading to production of |
| | | RAGE binding | proinflammatory cytokines and up-regulation of cell |
| | | protein | adhesion molecules ICAM1 and VCAM1. Acts as a |
| | | S100 calcium- | monocyte and mast cell chemoattractant. Can stimulate |
| | | binding protein | mast cell degranulation and activation which generates |
| | | A12 | chemokines, histamine and cytokines inducing further |
| | | | leukocyte recruitment to the sites of inflammation. Can |
| | | | inhibit the activity of matrix metalloproteinases; |
| | | | MMP2, MMP3 and MMP9 by chelating Zn²⁺ from |
| | | | their active sites (By similarity). |
| 9. | Q28050 | Protein S100- | Bos Taurus (Bovine) |
| | | A7 | Causes an allergic reaction in human. Minor allergen |
| | | Calcium-binding | of bovine dander. |
| | | protein in | Metal binding (Zn, Fe), |
| | | amniotic fluid 2 | |
| | | Dander minor | |
| | | allergen BDA11 | |
| | | Dermal allergen | |
| | | BDA11 | |
| | | S100 calcium- | |
| | | binding protein | |
| | | A7 | |
| | | Allergen Bos d3 | |
| 10. | P28782 | Protein S100- | Bos Taurus (Bovine) |
| | | A8 | S100A8 is a calcium- and zinc-binding protein |
| | | BEE11 | which plays a prominent role in the regulation of |
| | | Calgranulin-A | inflammatory processes and immune response. It can |
| | | Neutrophil | induce neutrophil chemotaxis and adhesion. |
| | | cytosolic 7 kDA | Predominantly found as calprotectin (S100A8/A9) |
| | | protein | which has a wide plethora of intra- and extracellular |
| | | S100 calcium- | functions. The intracellular functions include: |
| | | binding protein | facilitating leukocyte arachidonic acid trafficking and |
| | | A8 | metabolism, modulation of the tubulin-dependent |
| | | | cytoskeleton during migration of phagocytes and |
| | | | activation of the neutrophilic NADPH-oxidase. |
| | | | Activates NADPH-oxidase by facilitating the enzyme |
| | | | complex assembly at the cell membrane, transferring |
| | | | arachidonic acid, an essential cofactor, to the enzyme |
| | | | complex and S100A8 contributes to the enzyme |
| | | | assembly by directly binding to NCF2/P67PHOX. The |
| | | | extracellular functions involve proinflammatory, |
| | | | antimicrobial, oxidant-scavenging and apoptosis- |
| | | | inducing activities. Its proinflammatory activity |
| | | | includes recruitment of leukocytes, promotion of |
| | | | cytokine and chemokine production, and regulation of |
| | | | leukocyte adhesion and migration. Acts as an alarmin |
| | | | or a danger associated molecular pattern (DAMP) |
| | | | molecule and stimulates innate immune cells via |
| | | | binding to pattern recognition receptors such as Toll- |
| | | | like receptor 4 (TLR4) and receptor for advanced |
| | | | glycation endproducts (AGER). Binding to TLR4 and |
| | | | AGER activates the MAP-kinase and NF-kappa-B |
| | | | signaling pathways resulting in the amplification of the |
| | | | proinflammatory cascade. Has antimicrobial activity |
| | | | towards bacteria and fungi and exerts its antimicrobial |
| | | | activity probably via chelation of Zn2+ which is |
| | | | essential for microbial growth. Can induce cell death |
| | | | via autophagy and apoptosis and this occurs through |
| | | | the cross-talk of mitochondria and lysosomes via |
| | | | reactive oxygen species (ROS) and the process |
| | | | involves BNIP3. Can regulate neutrophil number and |
| | | | apoptosis by an anti-apoptotic effect; regulates cell |
| | | | survival via ITGAM/ITGB and TLR4 and a signaling |
| | | | mechanism involving MEK-ERK. Its role as an |
| | | | oxidant scavenger has a protective role in preventing |
| | | | exaggerated tissue damage by scavenging oxidants (By |
| | | | similarity). The iNOS-S100A8/A9 transnitrosylase |
| | | | complex is proposed to direct selective inflammatory |
| | | | stimulus-dependent S-nitrosylation of multiple targets |
| | | | such as GAPDH, ANXA5, EZR, MSN and VIM by |
| | | | recognizing a [IL]-x-C-x-x-[DE] motif; S100A8 seems |
| | | | to contribute to S-nitrosylation site selectivity (By |
| | | | similarity). |
| 11. | A0JNP2 | Secretoglobin | Bos Taurus (Bovine) |
| | | family 1D | May bind androgens and other steroids. May be |
| | | member | under transcriptional regulation of steroid hormones |
| | | | (By similarity) |
| | | | Belongs to the secretoglobin family. Lipophilin |
| | | | subfamily. |
| 12. | P93693 | Serpin-Z1B | Triticum aestivum (Wheat) |
| | | TriaeZ1b, | Inhibits chymotrypsin and cathepsin G in vitro. |
| | | WSZ1b, WZS2 | Protease inhibitor, Serine protease inhibitor |
| | | Tri a 33 | |
| 13. | Q8SPP7 | Peptidoglycan | Bos Taurus (Bovine) |
| | | recognition | Innate immunity protein that plays several important |
| | | protein 1 | functions in antimicrobial and antitumor defense |
| | | PGLYRP1 | systems. Acts as a pattern receptor that binds to |
| | | Oligosaccharide- | murein peptidoglycans (PGN) of Gram-positive |
| | | binding protein | bacteria and thus provides bactericidal activity. Forms |
| | | (OBP) | an equimolar complex with heat shock protein |
| | | Peptidoglycan | HSPA1A and induces programmed cell death through |
| | | recognition | apoptosis and necroptosis in tumor cell lines by |
| | | protein short | activating the TNFR1 receptor on the target cell |
| | | (PGRP-S) | membrane. In addition, acts in complex with the |
| | | | Ca²⁺-binding protein S100A4 as a chemoattractant |
| | | | able to induce lymphocyte movement. |
| | | | Mechanistically, this complex acts as a ligand of the |
| | | | chemotactic receptors CCR5 and CXCR3 which are |
| | | | present on the cells of the immune system. Promotes |
| | | | also the activation of lymphocytes that become able to |
| | | | kill virus-infected cells as well as tumor cells by |
| | | | modulating the spectrum of their target-cell specificity. |
| | | | Induction of cytotoxicity on monocyte surface requires |
| | | | interaction with TREM1 receptor (By similarity). |

Most of the proteins in the table above have carrier function and may have a ligand or be present in a complex with another protein or with a different molecule, such as, for example, a carbohydrate, fatty acid or other compound. Of the 13 proteins identified, 2 were lipocalins (the allergen Bos D2 and odorant-binding protein) and 6 were transport proteins (serum albumin, NPC intracellular cholesterol transporter, protein S100-A7/8/12, peptidoglycan recognition protein). Of the 13 proteins, 11 are from cow (Bos taurus) and 2 are from wheat (Triticum aestivum).

The proteins or peptides derived from one of the proteins may comprise or consist of the exact amino acid sequence of any one of SEQ ID NO:1-13 or may comprise or consist of an amino acid sequence at least 90%, at least 95%, at least 99% or at least any value between 90% and 100% identical to any one of SEQ ID NO:1-13. Preferably, the proteins or peptides derived from one of the proteins may comprise or consist of the exact amino acid sequence of SEQ ID NO:2 or may comprise or consist of an amino acid sequence at least 90%, at least 95%, at least 99% or at least any value between 90% and 100% identical to SEQ ID NO:2. Also preferably, the proteins or peptides derived from one of the proteins may comprise or consist of the exact amino acid sequence of SEQ ID NO: 7 or may comprise or consist of an amino acid sequence at least 90%, at least 95%, at least 99% or at least any value between 90% and 100% identical to SEQ ID NO:7. The proteins may be part of a larger protein or may be in a complex with a ligand which may be another protein or a chemical molecule. The ligand is preferably a fatty acid. In some embodiments, the ligand is not a siderophore. The peptides may have a length of more than 10, 20, 30, 40, 50, 60, 70, 80, 90, 100 or more amino acids, wherein every value in between is included (for example 101, 95, 43).

The proteins or peptides derived from one of the proteins comprised in the compositions of the invention may be produced by standard recombinant means or may be isolated from barn dust extracts from cow sheds. In addition, the compositions of the invention may comprise further substances commonly found in barn dust extracts from cow sheds, such as arabinogalactan, bacterial metabolites or bacterial cell wall components.

Preferably, the protein or peptide derived from one of the proteins comprised in the compositions of the invention is produced recombinantly. As such, the protein or peptide is produced starting from a nucleic acid coding for the protein or peptide, preferably by means of genetic engineering methods. The production method can be carried out in vivo, the protein or peptide, can, for example, be produced in a bacterial or eukaryotic host organism and optionally then isolated from this host organism or its culture. It is also possible to produce a protein in vitro, for example by use of an in vitro translation system.

When producing a protein or peptide in vivo, a nucleic acid encoding such protein or peptide is introduced into a suitable bacterial or eukaryotic host organism, preferably using recombinant DNA technology (as already outlined above). For this purpose, a host cell is first transformed with a cloning vector that includes a nucleic acid molecule encoding a protein or peptide as described herein, for example, using established standard methods. The host cell is cultured under conditions, which allow expression of the heterologous DNA and thus the synthesis of the corresponding protein or peptide. Subsequently, the protein or peptide is preferably recovered either from the cell or the cultivation medium.

The term “barn dust” as used herein, relates to dust that can be, is, or has been collected from a barn. Without wishing to be bound by theory it is believed that barn dust comprises immunostimulatory substances derived from microorganisms, animals, plants, fungi, viruses and/or protozoa that are protective against allergies, asthma and/or other diseases disclosed herein. In preferred embodiments, the barn dust is from a farm. Dust from cow barns are however preferred. Also, the geographic location of the barn is believed to be not essential for the invention. Exemplary non-limiting geographic locations for a barn are Europe, including the member states of the European Union, such as Germany, France, Austria, Czech Republic, Poland, the Netherlands, Belgium, Luxemburg, Spain, Portugal, Italy, etc., or Switzerland, the barn may for example be located in Bavaria, Germany. As a non-limiting example, barn dust may be obtained from Hechfellner Hof, Mettenheim, Bavaria, Germany. Barn dust can be collected by any suitable method known to the person skilled in the art optionally by applying any type of collection system that is suitable for collecting barn dust. Barn dust can for example be collected by sweeping, vacuuming, or swiping. Barn dust can also be collected by filtration of barn air, for example by using the membrane filter or a granular material that is capable of adsorbing barn dust. Barn dust can also be collected using an impinge or an impactor, such as a cascade impactor.

After collection, the dust may be homogenized. A suitable type of technique is any technique which leads to a uniform homogenization of the dust, in particular one which crushes agglomerations and lumps of the dust. Suitable for these purposes are methods such as rubbing, smashing or crushing, stirring or introducing into a blender, without being limited thereto. Digesting the dust may also be part of the process according to the invention. Digesting of the constituents present in the dust, such as cells, microorganisms, in particular their spores and the like can be effected, for example by grinding, squashing and similar methods. A sieving step for removing (large) particles, such as particles having a size of about 100 μm, about 90 μm, about 80 μm, about 70 m, about 60 μm, about 50 μm, about 40 m, about 30 μm, about 20 μm, or about 10 μm may be applied before or after homogenization.

The term “barn dust extract” or “shed dust extract” as used herein preferably refers to a composition that is obtainable by the methods disclosed herein and may refer to both, a solution or suspension, or a dry composition.

The term “fraction” as used herein preferably refers to fractions that can be obtained by fractioning a mixture according to the molecular weight and/or size of the molecules comprising the mixture.

The term “isolating” or “isolation” as used herein preferably refers to enriching a portion that is to be isolated as compared to a portion that is not to be isolated by mass, or depleting a portion that is not to be isolated as compared to a portion that is to be isolated by mass. The term “consists essentially of” means that further components, such as impurities, can be present but that the further components do not markedly affect the essential characteristics of the components that the mixture, fraction, compound etc. essentially consists of. Such further component(s) may amount to up to about 10%, up to about 9%, up to about 8%, up to about 7%, up to about 6%, up to about 5%, up to about 4%, up to about 3%, up to about 2%, up to about 1%, up to about 0.5%, up to about 0.2%, or up to about 0.1% by weight of component(s) that the mixture, fraction, compound etc. essentially consists of. Preferably, a protein or peptide comprised in the compositions of the invention is an isolated protein or peptide. In this context, “isolated” protein or peptide refers to a protein or peptide that is substantially free of its natural environment. For instance, an isolated protein or peptide is substantially free of cellular material and other proteins from the cell or tissue source from which it is derived, or other barn dust components or other sources from which it is isolated.

The inventors of the present application previously found that isolating a fraction, in which the barn dust comprised therein consists essentially of molecules having a molecular weight of at least about 5 kDa, preferably at least about 10 kDa, will yield a highly standardized product that is essentially independent from the origin of the starting material (WO2020/120724).

The present invention also relates to a pharmaceutical composition comprising one or several of the proteins or peptides derived from these proteins of the present invention.

Such a composition may be a pharmaceutical composition that comprises the proteins or peptides derived from the proteins of the invention, or the barn dust extract as active ingredient and optionally, one or more pharmaceutically excipient(s). Accordingly, the use of a barn dust extract described herein, for the manufacture of a pharmaceutical composition or medicament is also envisaged herein.

The term “pharmaceutical composition” particularly refers to a composition suitable for administering to a human. However, compositions suitable for administration to non-human animals are generally also encompassed by the term.

The pharmaceutical composition and its components (i.e. active agents and optional excipients) are preferably pharmaceutically acceptable, i.e. capable of eliciting the desired therapeutic effect without causing any undesirable local or systemic effects in the recipient. Pharmaceutically acceptable compositions of the invention may for instance be sterile or non-sterile. Specifically, the term “pharmaceutically acceptable” may mean approved by a regulatory agency or pharmacopoeia for use in animals, and more particularly in humans.

Preferably, the pharmaceutical composition comprises the protein or peptide derived from the proteins of the invention in form of an isolated protein or peptide. Preferably, the pharmaceutical composition comprises the protein or peptide derived from the proteins of the invention in form of recombinantly produced protein or peptides. Preferably, the protein or peptide derived from the proteins of the invention are essentially free of impurities that are typically contained in barn dust extract, barn dust extract fractions, or barn dust extract isolates. The pharmaceutical composition is preferably essentially free of barn dust extract components other than the protein(s) or peptide(s) derived from the proteins of the invention.

The proteins or peptides derived from the proteins of the invention are preferably present in the pharmaceutical composition in a therapeutically effective amount. By “therapeutically effective amount” is meant an amount of the active agent that elicits the desired therapeutic or prophylactic effect. Therapeutic efficacy can be determined by standard procedures, e.g. in test animals, expressed e.g., as ED₅₀ (the dose therapeutically effective in 50% of the population).

The term “excipient” includes fillers, binders, disintegrants, coatings, sorbents, antiadherents, glidants, preservatives, antioxidants, flavoring, coloring, sweeting agents, solvents, co-solvents, buffering agents, chelating agents, viscosity imparting agents, surface active agents, diluents, humectants, carriers, diluents, preservatives, emulsifiers, stabilizers, tonicity modifiers etc. It is within the knowledge of the skilled person to select suitable excipients for preparing the desired pharmaceutical composition of the invention. Exemplary carriers for use in the pharmaceutical composition of the invention include saline, buffered saline, dextrose, and water. Typically, choice of suitable excipients will inter alia depend on the specific active agent used, the disease to be treated, and the desired formulation of the pharmaceutical composition.

The exact dosage of the proteins described herein (or the pharmaceutical composition comprising the same), will be ascertainable by one skilled in the art using known techniques. Suitable dosages provide sufficient amounts of the proteins and are preferably therapeutically effective.

The compositions of the invention can be formulated in various forms, e.g. in solid, liquid, gaseous or lyophilized form and may be, for instance, in the form of a solution, an aerosol, a suspension, a lyophilisate, a powder, a tablet, a dragee, a suppository, enema, a pill, a capsule, granule, an ointment, a cream, a plaster, transdermal patches, a gel, suspensions, emulsions, syrups, liquids, elixirs, extracts, tincture or fluid extracts or in a form which is particularly suitable for the desired method of administration.

A variety of routes are applicable for administration of the composition according to the present invention. Typically, the composition may be prepared for nasal, inhalative, oral, conjunctival, dermal, subcutaneous, intraarticular, intraperitoneal, rectal, or vaginal administration. The composition may be in a form of a food additive, a food ingredient, or a composition suitable to be distributed in indoor air.

A composition comprising the proteins or peptides derived from the proteins of the invention or a pharmaceutical composition of the invention, may be for use in the prevention or treatment of a disease. The use preferably comprises administering the composition to a subject that is preferably in need thereof. A pharmaceutical composition of the invention may be used for the prevention or treatment of a disease. The present invention also contemplates a method of preventing or treating a disease comprising administering a therapeutically effective amount of a pharmaceutical composition of the invention to a subject.

The term “treatment” in all its grammatical forms includes therapeutic or prophylactic treatment of a subject in need thereof. A “therapeutic or prophylactic treatment” comprises prophylactic treatments aimed at the complete prevention of clinical and/or pathological manifestations or therapeutic treatment aimed at amelioration or remission of clinical and/or pathological manifestations. The term “treatment” thus also includes the amelioration or prevention of diseases.

The disease may be selected from the group consisting of an allergic disease, a chronic inflammatory disease, and an autoimmune disease. Preferably, the disease is selected from the group consisting of hay fever, food allergy, asthma, urticaria, neurodermitis, atopy, including atopic sensitisation and atopic dermatitis, contact eczema, psoriasis, diabetes type 1 or 2, multiple sclerosis, rheumatoid arthritis, diseases of the thyroid gland, including Hashimoto Thyreoditis and Graves disease, preferably selected from the group consisting of atopy, including atopic sensitisation and atopic dermatitis, asthma and hay fever.

It is envisioned by the invention that the “subject” may be an animal, preferably a vertebrate, preferably a mammal. Preferred subjects include human, mouse, rat, rabbit, hamster, guinea pig, pig, dog, cat, cattle, sheep, goat, horse, camel, monkey, or ape. It is envisioned by the invention that a human subject is preferred over other species, wherein a baby, and infant, or a pregnant woman is most preferred.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range. It includes, however, also the concrete number, e.g., about 20 includes 20.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent applications, scientific publications, manufacturer's specifications, instructions, etc.) are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

### EXAMPLES

### Materials and Methods

### Shed Dust Extract (SDE) Preparation

European farm dust was collected from stables of traditional European farms, which mainly housed cows or sheep and stored at −20° C. before use. For extraction 2.5 μg of farm dust (particle size 40-100 m) was weighted into a 50 mL centrifuge tube, 25 mL of sterile water was added and shaken for 2 h at 1,000 rpm at RT. Next, the sample was centrifuged at 2,500 μg for 5 min at RT. First, the supernatant was filtrated through two bottle-top filtration systems (pore size 13 μm and 2 m; cellulose membrane) to remove insoluble material. Then the prefiltered extract was filtered a third time through a bottle-top filtration system (pore size 0.22 m polyethersulfone (PES) membrane). After the filtration steps the SDE was autoclaved with pressurized saturated steam for 15 min at 121° C. Then the sterile SDE was centrifuged with 20 mL centrifugal concentrators (PES membrane, molecular weight cut off (MWCO) 10 kDa; Sartorius, Göttingen, Germany) at 3,500 μg for 45 min at RT. Ten millilitres of sterile water was added to the obtained solid residue, containing molecules with a MWCO of >10 kDa. The water was removed by centrifuging at 3,500 μg for 30 min at RT (loss of restrained small molecules; purification step). The purification step was repeated under the same conditions before the precipitate was resuspended in sterile water. The resuspended SDE (MWCO>10 kDA) was filtered a fourth time through a bottle top filtration system (0.22 m PES membrane). Finally under sterile conditions, 3.0 mL of the sterile and concentrated SDE was filled into a 5 mL glass vial and freeze-dried. The lyophilized farm dust extract was stored at −20° C.

### Example 1

### Nuclear Magnetic Resonance Spectroscopy

A Bruker Avance III spectrometer and TopSpin 3.2/PL7 software were used to acquire one-dimensional ¹H nuclear magnetic resonance (NMR) spectra of re-dissolved SDE. Proton-detected NMR spectra of SDE were acquired at 800.13 MHz (B₀=18.7 T) and 300 K with a 5-mm z-gradient ¹H/¹³C/¹⁵N/³¹P QCI cryogenic probe (quaternary cryogenic inverse; 90° excitation pulses: ¹³C˜¹H˜10 μs). 1-4 mg of SDE were dissolved in ˜40-130 mg D₂O (Merck, ²H: 99.96%), and centrifuged for 5 min at 4,000 rpm. The supernatant (brownish transparent solutions) was transferred into Bruker Match NMR tubes (1.7-3.0 mm) and sealed. NMR chemical reference δ_{H}: (H₃C)₃Si—CD₂CD₂-COOH: 0 ppm. 1-D ¹H NMR spectra were recorded with a spin-echo sequence (10 μs delay) to allow for high-Q probe ring-down (Q: high quality factor), and classical presaturation to attenuate residual water present: noesyprld, typically 128-1024 scans (5 s acquisition time, 5 s relaxation delay (d1), 1 ms mixing time; 1 Hz exponential line broadening).

Results are shown in FIGS. 1 and 2. Specifically, ¹H-NMR spectra of cow barn dust extract and sheep barn dust extract can be seen. The barn dust extracts of cow sheds were found to be high in peptides while the extracts from sheep sheds were higher in carbohydrates.

### Example 2

### Mass Spectrometric Analysis of Shed Dust Extracts

### Samples Measurement 1

### Autoclaved Samples (+):

Sheep-01_>10+(Sheep)
Cow-01-year-20>10+(Cow)

### Samples Measurement 2

### Non-Autoclaved Samples (−):

| Nr. Sample Description | Species |
|---|---|
| 1. Cow-01-year-20 > 10- | Cow |
| 2. Cow-01-year-18 > 10- | Cow |
| 3. Cow-02 > 10- | Cow |
| 4. Cow-03 > 10- | Cow |
| 5. Cow-04 > 10- | Cow |
| 6. Sheep-01 > 10- | Sheep |
| 7. Sheep-02 > 10- | Sheep |

### Samples Measurement 3

### Autoclaved Samples (+)

| Nr. | Sample Description | Species |
|---|---|---|
| 1 | Cow-01-year-20 > 10 kDa (+) | Cow |
| 2 | Cow-02 > 10 kDa (+) | Cow |
| 3 | Cow-03 > 10 kDa (+) | Cow |
| 4 | Cow-04 > 10 kDa (+) | Cow |
| 5 | Cow-05 > 10 kDa (+) | Cow |
| 6 | Cow-06 > 10 kDa (+) | Cow |
| 7 | Cow-07 > 10 kDa (+) | Cow |
| 8 | Sheep-02 > 10 kDa (+) | Sheep |

### Sample Preparation for Mass Spectrometric Analysis

Dried SDE samples with or without previous autoclaving obtained as described in WO 2020/120724 and above were dissolved in 100 μl 50 mM ammoniumbicarbonate and incubated for 10 minutes at room temperature shaking followed by three rounds of ultrasound bath for 30 seconds. After addition of 100 μl 8M urea in 100 mM Tris-HCl, pH 8.5, and again incubation for 10 minutes at room temperature shaking followed by three rounds of ultrasound bath for 30 s, the samples were centrifuged for 15 minutes at 14,000 μg to remove unsolubilized proteins. Protein content in the supernatant was determined using the Bradford assay. 10 μg per sample were digested with Lys-C and trypsin using a modified FASP procedure (Wiśniewski et al., 2009, Universal sample preparation method for proteome analysis. Nat Methods 6, 359-362; Grosche et al., 2015, The proteome of native adult Muller glial cells from murine retina. Mol Cell Proteomics).

### LC-MSMS and Quantitative Analysis

LC-MSMS analysis was performed on a QExactive HF or HFX mass spectrometer (ThermoFisher Scientific) online coupled to a Ultimate 3000 RSLC nano-HPLC (Dionex). Samples were automatically injected and loaded onto the C18 trap column and after 5 min eluted and separated on the C18 analytical column (Acquity UPLC M-Class HSS T3 Column, 1.8 μm, 75 m×250 mm; Waters) by a 90 min non-linear acetonitrile gradient at a flow rate of 250 nL/min. MS spectra were recorded at a resolution of 60 000 with an AGC target of 3×10⁶ and a maximum injection time of 50 or 30 ms from 300 to 1500 m/z. From the MS scan, the 10 or 15 most abundant peptide ions were selected for fragmentation via HCD with a normalized collision energy of 27 or 28, an isolation window of 1.6 m/z, and a dynamic exclusion of 30 s. MS/MS spectra were recorded at a resolution of 15,000 with a AGC target of 10⁵ and a maximum injection time of 50 ms. Unassigned charges, and charges of +1 and >8 were excluded from precursor selection.

Acquired raw data for the three datasets (autoclaved pilot, non-autoclaved replicates, autoclaved replicates) were analyzed separately. The Proteome Discoverer 2.4 SPI software (Thermo Fisher Scientific; version 2.4.1.15) was used for peptide and protein identification via a database search (Sequest HT search engine) against the SwissProt All database (version 3.0, 474,326 sequences, 202,348,262 residues), considering full tryptic specificity, allowing for up to one missed tryptic cleavage sites, precursor mass tolerance 10 ppm, fragment mass tolerance 0.02 Da. Carbamidomethylation of Cys was set as a static modification. Dynamic modifications included deamidation of Asn and Gln, oxidation of Met, and a combination of Met loss with acetylation on protein N-terminus. The Percolator algorithm (Käll et al., 2007, Semi-supervised learning for peptide identification from shotgun proteomics datasets. Nat Methods 4, 923-925) was used for validating peptide spectrum matches and peptides, accepting only the top-scoring hit for each spectrum, and satisfying FDR<1% (high confidence). The final list of proteins satisfying the strict parsimony principle included only protein groups passing an additional protein confidence filter FDR<5% filter (target/decoy concatenated search validation).

Quantification of proteins, after precursor recalibration, was based on intensity values (at RT apex) for all unique peptides per protein. Peptide abundance values were not normalized. The protein abundances were calculated summing the raw abundance values of corresponding unique peptides. These raw protein abundances were used for calculation of enrichment ratios of proteins in the individual cow dust samples to the included sheep dust samples, resulting in single ratios for every quantified protein in every cow dust sample.

The three datasets were then combined, resulting in a list of 1888 identified proteins, of which 264 proteins fulfilled the criteria to be quantified with at least 2 peptides in at least one of the three datasets and show an enrichment ratio cow/sheep of >5 for at least one cow dust sample. As some of the 7 cow farms were measured repeatedly, we further filtered for an enrichment ratio of >5 in at least one sample per dataset and for at least 5 of the 7 included farms, resulting in the below list of 13 proteins.

| | | Measurement 2 | Measurement 3 |
|---|---|---|---|
| | Measurement 1 | (non-autoclaved | (autoclaved |
| | (autoclaved) | samples) | samples) |
| Accession | Identified unique peptides | | |
| P02769 | 22 | 17 | 13 |
| Q28133 | 20 | 13 | 20 |
| P79124 | 9 | 2 | 9 |
| P80416 | 2 | 1 | 2 |
| P01083 | 4 | 1 | 4 |
| P79345 | 1 | 1 | 4 |
| P07435 | 9 | 4 | 10 |
| P79105 | 8 | 1 | 6 |
| Q28050 | 9 | 7 | 12 |
| P28782 | 5 | 2 | 4 |
| A0JNP2 | 2 | 1 | 2 |
| P93693 | 4 | 1 | 4 |
| Q8SPP7 | 6 | 1 | 7 |

Analysis of these proteins showed that most of the proteins have carrier functions and may be active in combination with a ligand.

| Accession | Name |
|---|---|
| P02769 | Serum albumin OS = Bos taurus OX = 9913 GN = ALB PE = 1 SV = 4 |
| Q28133 | Allergen Bos d 2 OS = Bos taurus OX = 9913 PE = 1 SV = 1 |
| P79124 | Short palate, lung and nasal epithelium carcinoma-associated |
| | protein 2A OS = Bos taurus OX = 9913 GN = SPLUNC2A PE = 2 SV = 2 |
| P80416 | Cystatin-A OS = Bos taurus OX = 9913 GN = CSTA PE = 1 SV = 1 |
| P01083 | Alpha-amylase inhibitor 0.28 OS = Triticum aestivum OX = 4565 |
| | GN = IMA1 PE = 1 SV = 3 |
| P79345 | NPC intracellular cholesterol transporter 2 OS = Bos taurus |
| | OX = 9913 GN = NPC2 PE = 1 SV = 1 |
| P07435 | Odorant-binding protein OS = Bos taurus OX = 9913 PE = 1 SV = 2 |
| P79105 | Protein S100-A12 OS = Bos taurus OX = 9913 GN = S100A12 PE = 1 |
| | SV = 3 |
| Q28050 | Protein S100-A7 OS = Bos taurus OX = 9913 GN = S100A7 PE = 1 |
| | SV = 1 |
| P28782 | Protein S100-A8 OS = Bos taurus OX = 9913 GN = S100A8 PE = 1 |
| | SV = 2 |
| A0JNP2 | Secretoglobin family 1D member OS = Bos taurus OX = 9913 |
| | GN = SCGB1D PE = 3 SV = 2 |
| P93693 | Serpin-Z1B OS = Triticum aestivum OX = 4565 PE = 1 SV = 1 |
| Q8SPP7 | Peptidoglycan recognition protein 1 OS = Bos taurus OX = 9913 |
| | GN = PGLYRP1 PE = 1 SV = 1 |

The next step will be to identify potential ligands and test the effectiveness of the proteins alone and with the ligand.

### Example 3

### Sequences of Recombinant Expressed Proteins:

His-Trx-bOPB:
(SEQ ID NO: 14)
MKHHHHHHPMSDKIIHLTDDSFDTDVLKADGAILVDFWAEWCGPCKMIA
PILDEIADEYQGKLTVAKLNIDQNPGTAPKYGIRGIPTLLLFKNGEVAA
TKVGALSKGQLKEFLDANLAGSGSGSENLYFQGAMAQEEEAEQNLSELS
GPWRTVYIGSTNPEKIQENGPFRTYFRELVFDDEKGTVDFYFSVKRDGK
WKNVHVKATKQDDGTYVADYEGQNVFKIVSLSRTHLVAHNINVDKHGQT
TELTELFVKLNVEDEDLEKFWKLTEDKGIDKKNVVNFLENEDHPHPE
His-Trx-Bos d2:
(SEQ ID NO: 15)
MKHHHHHHPMSDKIIHLTDDSFDTDVLKADGAILVDFWAEWCGPCKMIA
PILDEIADEYQGKLTVAKLNIDQNPGTAPKYGIRGIPTLLLFKNGEVAA
TKVGALSKGQLKEFLDANLAGSGSGSENLYFQGAMMKAVFLTLLFGLGL
VCTAQETPAEIDPSKIPGEWRIIYAAADNKDKIVEGGPLRNYYRRIECI
NDCESLSITFYLKDQGTCLLLTEVAKRQEGYVYVLEFYGTNTLEVIHVS
ENMLVTYVENYDGERITKMTEGLAKGTSFTPEELEKYQQLNSERGVPNE
NIENLIKTDNCPP

Recombinant Expression and Purification of Bos d2 and bOBP:

E. coli BL21 cells were transformed with plasmid petTRX-Bos d2 or petTRX-OBP encoding sequences of Bos d2 or bOBP with N-terminal 6×His-Thioredoxin tag and a TEV cleavage site. Proteins expression was performed over-night at 20° C. in TB broth medium (and in case of ¹⁵N-labeled bOBP in M9 minimal medium) with additional 100 μg/ml Kanamycin. Harvested cells were lysed by sonication and lysate was initially purified by immobilized metal ion chromatography (IMAC). His-tagged TEV protease was added to combined elution fractions and samples were dialysed at 4° C. over-night. Cleaved tag and protease were removed by another IMAC and cleaved protein was applied to size exclusion chromatography (FIG. 3). Purified samples were shock-frozen and stored at −80° C. until further use.

### Preparation of Liporotein Complexes:

Bos d2 or bOBP were incubated for 1 h at 4° C. with 5 fold molar excess of fatty acid ((E)-12-oxoheptadec-10-enoic acid in DMSO-d6). Samples were dialysed (MWCO 3.5 kDa) over-night at 4° C. against phosphate-buffered saline (PBS) with 1 mM β-mercaptoethanol to remove unbound fatty acids, DSMO and other impurities of the SDE. Samples were concentrated to 1 mg/ml. Control samples with lipocalin without ligand were prepared in identical manner.

### Nuclear Magnetic Resonance (NMR) Binding Studies:

NMR measurements were performed in 3 mm tubes at 25° C. on a Bruker Avance III spectrometer operating at 600 MHz ¹H frequencies using H/N/C triple-resonance cryogenic probes. To confirm fatty acid binding, two-dimensional ¹H, ¹⁵N heteronuclear single quantum correlation (HSQC) spectra were recorded of 200 μM ¹⁵N-bOBP in 20 mM phosphate buffer pH 6.5, 50 mM NaCl, 1 mM β-mercaptoethanol and 10% (v/v) D₂0 alone or in presence of 1 mM and 2 mM fatty acid. Both titration experiments show chemical shift perturbations (CSP) which is indicative for ligand binding (FIG. 4).

### Activation of TLR-2

We have identified that toll-like receptor 2 (TLR2) is activated by the autoclaved>10 kDa European cow SDE. In human TLR2 transfected HEK cells the extract was almost as active as its positive control Pam3Cys. Of note, no activation of TLR4 was seen which points to the absence of a relevant LPS effect.

HEK-Blue™-hTLR2 cells (InvivoGen) are obtained by co-transfection of the human TLR2 and SEAP (secreted embryonic alkaline phosphatase) genes into HEK293 cells. The SEAP reporter gene is placed under the control of the IFN-β minimal promoter fused to five NF-κB and AP-1-binding sites. Additionally, the CD14 co-receptor gene was transfected into these cells to enhance the TLR2 response. Stimulation with a TLR2 ligand activates NF-κB and AP-1, which in turn induce the production of SEAP. Levels of SEAP are determined with HEK-Blue™ Detection, a cell culture medium that allows for real-time detection of SEAP. The recombinant Proteins Bos d2 and OBP bound to the fatty acid (E)-12-oxoheptadec-10-enoic acid did show an even stronger effect than the cow SDE (>10 kDa, autoclaved) alone. OBP alone had an effect as well, but higher concentrations are needed as compared to OBP bound to the fatty acid (FIG. 5).

### TEER Assay

To examine the effect of the Lipoproteins Bos d2+FA and OBP+FA on the epithelial barrier function as measured by transepithelial electrical resistance (TEER). 16HBE (100.000 (1×10*5)) cells were seeded on transwell inserts in serum-free medium (MEM with Earle's salt, 10% FBS (during cultivation only, during assay no FBS), 1% L-Glutamine, 1% Penicillin/Streptomycin). After 24 hours of culture, cells were treated with the indicated compounds, and 24 hours after treatment, TEER values were determined using an EVOM2 epithelial voltohmmeter (World Precision Instruments). Cow SDE was used at 200 μg/ml, all other compounds were used at 20 μg/ml. Again, the Lipoproteins Bos d2+FA and OBP+FA had a stronger effect, at much lower concentration as the original cow shed dust extract (>10 kDA, autoclaved) (FIG. 6).

### Example 4

In vitro Assay:

### DC/T-Cell Interaction:

DCs are professional antigen presenting cells crucial for the priming of CD4 T cells in asthma and allergy. The mouse primary bone marrow derived DCs (BMDCs) were utilized to evaluate the ability of the test substances to modulate their activation. DC activation is monitored their ability to regulate the differentiation and proliferation of OVA-specific CD4 T cells (OT-II) following DC pulsing with the OVA peptide.

### The Assay Set-Up is Shown in FIG. 7

For this assay, bone marrow derived DCs are isolated from C57B16 mice and put into culture. After treatment with farm dust extract (or shame treatment) the DCs are pulsed with Ova peptide and are then cocultured with CD4+ T-cells from OTII mice (presensitized to OVA).

### DETAILED DESCRIPTION

Isolate bone marrow from wild-type C57BL/6 mouse and seed cells in 10 ml Petri dishes in 10 ml volume (2 million cells/dish) containing 20 ng/ml rmGM-CSF. Add 10 ml fresh media on D3 containing 40 ng/ml rmGM-CSF bringing the final volume to 20 ng/ml. On D6 and D8, half of the media was replaced with fresh medium keeping the final GM-CSF concentration at 20 ng/ml. Half of the medium was removed from the dishes and centrifuged to replace the medium. The pellet was then resuspended with fresh medium and added to the dishes. On D10, half of the medium was replaced but with halved rmGM-CSF concentration. On D12, all non-adherent and loosely attached cells were collected and spun down at 410 g for 10 mins. Cells were resuspended with 10 ml fresh media/dish (+10 ng/ml rmGM-CSF) also containing the respective treatment (cow dust: 100 ug/ml). On D13, all medium was collected, centrifuged, and the cell pellet was resuspended with the medium containing the treatment for the second dose. Treatment was initiated for the single treatment group on this day as well. On D14, LPS was added (1 ug/ml final concentration) to cells and incubated for 6 hours. Cells were then collected and counted with the help of counting beads and seeded in V bottom 96 well plates (10000 cells/well) and pulsed with OVA peptide (323-339) for 3 hours. In the meantime, naïve CD4+ T Cells were isolated from OTII splenocytes following Miltenyi's isolation protocol, and cells were stained with CTV to follow proliferation. 20000 stained T Cells were cocultured with DCs for 72 days, and T Cells were then analyzed with flow cytometry.

The following substances were tested and compared:

$$Bosd 2 + FA = His - Trx - Bosd 2 + ( E ) - 12 - oxoheptadec - 10 - enaic acid ( 10 ug / ml )$$

$$OBP + FA = bOBP + ( E ) - 12 - oxoheptadec - 10 - enaic acid ( 10 ug / ml )$$

Results of T-cell profilferation after two treatments with the respective test substance and after stimulation with LPS are shown in FIG. 8.

### Example 5

### The Assay Set-Up is Shown in FIG. 9

Balb/c mice were first be sensitized with 10 μg OVA in Alum i.p. on day 0, 7 and 14. Three consecutive intranasal challenges with 100 μg OVA on day 25, 26 and 27, with analysis being undertaken on day 28, follows. From d0-d26 there were 12 consecutive intranasal applications of test substance. Asthma pathogenesis was assessed using methacholine lung function challenge to examine AHR as undertaken in allergic asthma patients. Furthermore, pathological manifestations such as eosinophilia was assessed.
Sensitzation: 10 μg valbumin (OVA Grade VI, Sigma, Stainheim, D) dissolved in 100 μl PBS on days 0, 7 and 14 of the assay protocol together with 100 μl (1 mg) of the adjuvant aluminium hydroxyde (Al(OH)₃) (Inject Alum, Pierce, USA).
Challenge: OVA Grade V in 100 μg/mouse in 25 μl (concentration: 4 mg/ml)

Eosiniphilnumbers in the Bronchoalveolar lavage (BAL) are shown in FIG. 10.
Airway Hyperresponsivenes (AHR): Dynamic resistance and compliance (in response to increasing doses of MCh) FlexiVent results are shown in FIG. 11.

OBP+FA as well as OBP alone show a significant effect on Eosinophils and AHR. For AHR the effect of OBP+FA is slightly better than for OBP alone. The FA alone does not have an effect on eosinophils, but an effect on AHR.

1. A composition for use in the prevention or treatment of a disease comprising at least one protein or peptide derived from one of the proteins having an amino acid sequence at least 90% identical to any one of SEQ ID NO:1-13.

2. The composition for the use of claim 1, wherein the disease is selected from the group consisting of an allergic disease, a chronic inflammatory disease, and an autoimmune disease.

3. The composition for the use of claim 1 or 2, wherein the disease is selected from the group consisting of hay fever, food allergy, asthma, urticaria, neurodermitis, atopy, including atopic sensitisation and atopic dermatitis, contact eczema, psoriasis, diabetes type 1 or 2, multiple sclerosis, rheumatoid arthritis, diseases of the thyroid gland, including Hashimoto thyreoditis and Graves disease, preferably selected from the group consisting of atopy, including atopic sensitisation and atopic dermatitis, asthma and hay fever.

4. The composition for the use of any one of the preceding claims, wherein the composition comprises 2, 3, 4, 5, 6, 7, 8, 9, 10, 11, 12 or all of the proteins or peptides derived from one of the proteins having an amino acid sequence at least 90% identical to of any one of SEQ ID NO:1-13.

5. The composition for the use of any one of the preceding claims, wherein the proteins or peptides derived from one of the proteins are at least 95%, 99% or are 100% identical to any one of SEQ ID NO:1-13.

6. The composition for the use of any one of the preceding claims, wherein the composition comprises a combination of SEQ ID NO:1 together with at least one of SEQ ID NO:2-13.

7. The composition for the use of any one of the preceding claims, wherein the composition comprises a combination of SEQ ID NO:1 and SEQ ID NO:2, optionally with one or more of SEQ ID NO:3, SEQ ID NO:7 or SEQ ID NO:9.

8. The composition for the use of any one of the preceding claims, wherein the composition is in the form of a solution, an aerosol, a suspension, a lyophilisate, a powder, a tablet, a dragee, suppository, enema, ointment, creme or plaster.

9. The composition of any one of the preceding claims, wherein the composition is for nasal, inhalative, oral, conjunctival, dermal, subcutaneous, intraarticular, intraperitoneal, rectal, or vaginal administration.

10. The composition of any one of the preceding claims, wherein the at least one protein or peptide derived from one of the proteins having the amino acid sequence at least 90% identical to any one of SEQ ID NO:1-13 is bound to a ligand.

11. A pharmaceutical composition comprising at least one protein or peptide derived from one of the proteins having an amino acid sequence at least 90% identical to any one of SEQ ID NO:1-13.

12. The pharmaceutical composition of claim 11, wherein the composition comprises 2, 3, 4, 5, 6, 7, 8, 9, 10, 11, 12 or all of the proteins or peptides derived from one of the proteins having an amino acid sequence at least 90% identical to of any one of SEQ ID NO:1-13.

13. The pharmaceutical composition of any one claims 11 or 12, wherein the proteins are at least 95% or are 100% identical to any one of SEQ ID NO:1-13.

14. The pharmaceutical composition of any one of claims 11 to 13, wherein the composition comprises a combination of SEQ ID NO:1 together with at least one of SEQ ID NO:2-13.

15. The pharmaceutical composition for the use of any one of claims 11 to 14, wherein the composition comprises a combination of SEQ ID NO:1 and SEQ ID NO:2, optionally with one or more of SEQ ID NO:3, SEQ ID NO:7 or SEQ ID NO:9.

16. The pharmaceutical composition of any one of claims 11 to 15, wherein the composition is in the form of a solution, an aerosol, a suspension, a lyophilisate, a powder, a tablet, a dragee, suppository, enema, ointment, creme or plaster.

17. The pharmaceutical composition of any one of claims 11 to 16, wherein the composition is for nasal, inhalative, oral, conjunctival, dermal, subcutaneous, intraarticular, intraperitoneal, rectal, or vaginal administration.

18. The pharmaceutical composition of any one of the preceding claims, wherein the at least one protein or peptide derived from one of the proteins having the amino acid sequence at least 90% identical to any one of SEQ ID NO:1-13 is bound to a ligand.


SEQUENCE LISTING (15 sequences)

SEQ ID NO: 1 - protein, 607 aa, Bos taurus
MKWVTFISLLLLFSSAYSRGVFRRDTHKSEIAHRFKDLGEEHFKGLVLIAFSQYLQQCPFDEHVKLVNELTEFAKTCVADESHAGCEKSLHTLFGDELCKVASLRETYGDMADCCEKQEPERNECFLSHKDDSPDLPKLKPDPNTLCDEFKADEKKFWGKYLYEIARRHPYFYAPELLYYANKYNGVFQECCQAEDKGACLLPKIETMREKVLASSARQRLRCASIQKFGERALKAWSVARLSQKFPKAEFVEVTKLVTDLTKVHKECCHGDLLECADDRADLAKYICDNQDTISSKLKECCDKPLLEKSHCIAEVEKDAIPENLPPLTADFAEDKDVCKNYQEAKDAFLGSFLYEYSRRHPEYAVSVLLRLAKEYEATLEECCAKDDPHACYSTVFDKLKHLVDEPQNLIKQNCDQFEKLGEYGFQNALIVRYTRKVPQVSTPTLVEVSRSLGKVGTRCCTKPESERMPCTEDYLSLILNRLCVLHEKTPVSEKVTKCCTESLVNRRPCFSALTPDETYVPKAFDEKLFTFHADICTLPDTEKQIKKQTALVELLKHKPKATEEQLKTVMENFVAFVDKCCAADDKEACFAVEGPKLVVSTQTALA

SEQ ID NO: 2 - protein, 172 aa, Bos taurus
MKAVFLTLLFGLVCTAQETPAEIDPSKIPGEWRIIYAAADNKDKIVEGGPLRNYYRRIECINDCESLSITFYLKDQGTCLLLTEVAKRQEGYVYVLEFYGTNTLEVIHVSENMLVTYVENYDGERITKMTEGLAKGTSFTPEELEKYQQLNSERGVPNENIENLIKTDNCPP

SEQ ID NO: 3 - protein, 239 aa, Bos taurus
MVQLWKLVLLCGLLAGTSESLLDIRGNDVLRRLISGLERGLGTFDSTIEIIFQNLKTELESRCLNDVVEETQQTENSLEGLISRIFQVVNRLTGVRIRNVQVPDITFEATSENSADVSIPITADVTVSLPLLGEIVKLDLNVDLQTSVSIETDAETGDSRVVVGECPNNPESISLTVLHRRPGLLNDVVDFGVNLVRQLVSSVVQHELCPRIRELLESLDTECIKKLIGEPQVTTQQEI

SEQ ID NO: 4 - protein, 98 aa, Bos taurus
MIPGGLTEAKPATIEIQEIANMVKPQLEEKTNETYEEFTAIEYKSQVVAGINYYIKIQTGDNRYIHIKVFKSLPQQSHSLILTGYQVDKTKDDELAGF

SEQ ID NO: 5 - protein, 153 aa, Triticum aestivum
MWMKTVFWGLLVFMLVATTMAVEYGARSHNSGPWSWCNPATGYKVSALTGCRAMVKLQCVGSQVPEAVLRDCCQQLADINNEWCRCGDLSSMLRSVYQELGVREGKEVLPGCRKEVMKLTAASVPEVCKVPIPNPSGDRAGVCYGDWAAYPDV

SEQ ID NO: 6 - protein, 149 aa, Bos taurus
MRFLTVAFLFLALSASALAEPVKFKDCGSWVGVIKEVNVSPCPTQPCKLHRGQSYSVNVTFTSNTQSQSSKAVVHGIVMGIPVPFPIPESDGCKSGIRCPIEKDKTYNYVNKLPVKNEYPSIKVVVEWELTDDKNQRFFCWQIPIEVEA

SEQ ID NO: 7 - protein, 159 aa, Bos taurus
AQEEEAEQNLSELSGPWRTVYIGSTNPEKIQENGPFRTYFRELVFDDEKGTVDFYFSVKRDGKWKNVHVKATKQDDGTYVADYEGQNVFKIVSLSRTHLVAHNINVDKHGQTTELTELFVKLNVEDEDLEKFWKLTEDKGIDKKNVVNFLENEDHPHPE

SEQ ID NO: 8 - protein, 92 aa, Bos taurus
MTKLEDHLEGIINIFHQYSVRVGHFDTLNKRELKQLITKELPKTLQNTKDQPTIDKIFQDLDADKDGAVSFEEFVVLVSRVLKTAHIDIHKE

SEQ ID NO: 9 - protein, 101 aa, Bos taurus
MSSSQLEQAITDLINLFHKYSGSDDTIEKEDLLRLMKDNFPNFLGACEKRGRDYLSNIFEKQDKNKDRKIDFSEFLSLLADIATDYHNHSHGAQLCSGGNQ

SEQ ID NO: 10 - protein, 89 aa, Bos taurus
MLTDLECAINSLIDVYHKYSLKKGNYHAVYRDDLKQLLETECPKFMKKKDADTWFKELDINQDGGINFEEFLVLVIKVGLEAHEEIHKE

SEQ ID NO: 11 - protein, 102 aa, Bos taurus
MRLSVTALLVTLALCYYEANAIVCPTFAADLTEFFYFPDLLYRLSLAKYNAPPEAVAAKMEVKQCTDRFSVKNRLIITNILGKILLNCTVTDVKAVLNPSSA

SEQ ID NO: 12 - protein, 399 aa, Triticum aestivum
MATTLATDVRLSIAHQTRFALRLASTISSNPKSAASNAAFSPVSLHSALSLLAAGAGSATRDQLVATLGTGEVEGGHALAEQVVQFVLADASSAGGPRVAFANGVFVDASLLLKPSFQELAVCKYKAETQSVDFQTKAAEVTTQVNSWVEKVTSGRIKNILPSGSVDNTTKLVLANALYFKGAWTDQFDSYGTKNDYFYLLDGSSVQTPFMSSMDDDQYISSSDGLKVLKLPYKQGGDNRQFSMYILLPEAPGGLSSLAEKLSAEPDFLERHIPRQRVAIRQFKLPKFKISFGMEASDLLKCLGLQLPFSDEADFSEMVDSPMPQGLRVSSVFHQAFVEVNEQGTEAAASTAIKMVPQQARPPSVMDFIADHPFLFLLREDISGVVLFMGHVVNPLLSS

SEQ ID NO: 13 - protein, 190 aa, Bos indicus
MSRRYTPLAWVLLALLGLGAAQDCGSIVSRGKWGALASKCSQRLRQPVRYVVVSHTAGSVCNTPASCQRQAQNVQHYHVRERGWCDVGYNFLIGEDGLVYEGRGWNTLGAHSGPTWNPIAIGISFMGNYMHRVPPASALRAAQSLLACGAARGYLTPNYEVKGHRDVQQTLSPGDELYKIIQQWPHYRRV

SEQ ID NO: 14 - protein, 292 aa, synthetic construct
MKHHHHHHPMSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGSENLYFQGAMAQEEEAEQNLSELSGPWRTVYIGSTNPEKIQENGPFRTYFRELVFDDEKGTVDFYFSVKRDGKWKNVHVKATKQDDGTYVADYEGQNVFKIVSLSRTHLVAHNINVDKHGQTTELTELFVKLNVEDEDLEKFWKLTEDKGIDKKNVVNFLENEDHPHPE

SEQ ID NO: 15 - protein, 307 aa, synthetic construct
MKHHHHHHPMSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGSENLYFQGAMMKAVFLTLLFGLGLVCTAQETPAEIDPSKIPGEWRIIYAAADNKDKIVEGGPLRNYYRRIECINDCESLSITFYLKDQGTCLLLTEVAKRQEGYVYVLEFYGTNTLEVIHVSENMLVTYVENYDGERITKMTEGLAKGTSFTPEELEKYQQLNSERGVPNENIENLIKTDNCPP